(19) United States
(12) Patent Application Publication
(10) Pub. No.: US 2025/0359746 A1
(43) Pub. Date: Nov. 27, 2025

(54) APPARATUS FOR IMPROVED CLEANING OF MEDICAL DEVICES

(71) Applicant: Boston Scientific Scimed, Inc., Maple Grove, MN (US)

(72) Inventors: Daniela CESPEDES VALENZUELA, Bogotá D.C. (CO); Santiago GRAJALES LONDOÑO, Bogotá (CO); Andrés Felipe MANRIQUE MORENO, Bogotá D.C. (CO); Juan Pablo HERRERA MEZA, Bogotá D.C. (CO); Santiago Sánchez RENTERÍA, Bogotá D.C. (CO); Carolina VILLARREAL, Hopedale, MA (US)

(73) Assignee: Boston Scientific Scimed, Inc., Maple Grove, MN (US)

(21) Appl. No.: 19/213,767

(22) Filed: May 20, 2025

Related U.S. Application Data
(60) Provisional application No. 63/650,103, filed on May 21, 2024.

(51) Int. Cl. A61B 1/12; A46B 5/06; A46B 13/00; A46B 13/08; A46D 1/00; F03B 3/04; F03B 11/06; F03B 13/00

(52) U.S. Cl. CPC A61B 1/122; A46B 5/06; A46B 13/006; A46B 13/08; A46D 1/0207; F03B 3/04; F03B 11/06; F03B 13/00; A46B 2200/3013

(57) ABSTRACT

Disclosed solutions relate to cleaning devices for medical devices. For example, a cleaning device for cleaning a channel of a medical device includes a rod. The cleaning device further includes a connector at a proximal end of the rod. The cleaning device further includes a flexible cleaner positioned around the rod. The cleaning device further includes a ferrule configured to removably connect with the connector. The cleaning device further includes a pull member connected to the ferrule.

### CROSS-REFERENCE TO RELATED APPLICATIONS

This application claims the benefit of priority of U.S. Provisional Patent Application No. 63/650,103, filed on May 21, 2024, the entirety of which is incorporated herein by reference.

### TECHNICAL FIELD

Various aspects of this disclosure relate generally to devices for cleaning medical devices, including for example endoscopes.

### BACKGROUND

Endoscopes have attained great acceptance within the medical community as they provide a means for performing procedures with minimal patient trauma while enabling the physician to view the internal anatomy of the patient. Numerous endoscopes have been developed and categorized according to specific applications, such as cystoscopy, colonoscopy, bronchoscopy, upper GI endoscopy, and others.

An endoscope usually has an elongated tubular shaft, having a video camera or a fiber optic lens assembly at its distal end. Various surgical tools may be inserted through a working channel in the endoscope for performing different surgical procedures.

Cleaning of the working channel of the endoscope is therefore important. But existing techniques can sometimes result in insufficient cleaning. Accordingly, new techniques are needed.

### SUMMARY

Aspects of the disclosure relate to, among other things, systems, devices, for cleaning inner channels of medical devices, including for example endoscopes.

In some aspects, the techniques described herein relate to a cleaning device for cleaning a channel of a medical device, the cleaning device including: a rod; a bristles positioned around and oriented radially outward from the rod; a cover proximal of the bristles; a bearing radially inward of an inner surface of the cover; and a propeller coupled to the rod, positioned radially inward of the cover, and rotatable within the bearing.

In some aspects, the techniques described herein relate to a cleaning device, further including a flange and a post, wherein the post is affixed to the rod via the flange, and wherein the propeller includes an axle that is configured to receive the rod.

In some aspects, the techniques described herein relate to a cleaning device, wherein the bearing is affixed to an inner surface of the cover via one or more spokes.

In some aspects, the techniques described herein relate to a cleaning device, wherein the bristles are formed of polypropylene.

In some aspects, the techniques described herein relate to a cleaning device, wherein each of the bristles extends radially with a first diameter that is greater than a second diameter of the cover.

In some aspects, the techniques described herein relate to a cleaning device, wherein the rod includes one or more braided strands.

In some aspects, the techniques described herein relate to a cleaning device, further including a pull cord that extends proximally from the cover and a anchor points positioned on the cover, wherein the anchor points are connected to the pull cord.

In some aspects, the techniques described herein relate to a cleaning device, wherein the anchor points includes three anchor points, and wherein the pull cord includes three segments, wherein each segment is connected to a respective anchor point.

In some aspects, the techniques described herein relate to a cleaning device, wherein the propeller includes a nose at a proximal edge of the propeller.

In some aspects, the techniques described herein relate to a cleaning device, wherein the propeller is configured such that, when a force is applied in a proximal direction in a presence of a fluid, the propeller rotates, thereby causing the rod and the bristles to rotate.

In some aspects, the techniques described herein relate to a cleaning device, further including a tip at a distal end of the rod.

In some aspects, the techniques described herein relate to a cleaning device, wherein the tip is spherical and formed of rubber.

In some aspects, the techniques described herein relate to a cleaning device for cleaning a channel of a medical device, the cleaning device including: a rod; a connector at a proximal end of the rod; a flexible cleaner positioned around the rod; a ferrule configured to removably connect with the connector; and a pull member connected to the ferrule.

In some aspects, the techniques described herein relate to a cleaning device, further including a tip at a distal end of the rod, wherein the ferrule is fixedly attached to a connector screw, wherein the rod, the connector, and the flexible cleaner, are removably connected to the ferrule and the connector screw.

In some aspects, the techniques described herein relate to a cleaning device, wherein the flexible cleaner includes a helical coil attached to the rod, wherein the helical coil includes a first diameter at a proximal end of the rod and a second diameter at a distal end of the rod, wherein the second diameter is greater than the first diameter.

In some aspects, the techniques described herein relate to a cleaning device, wherein the ferrule is attached to a connector screw, and wherein the connector includes one or more teeth that interface with the connector screw.

In some aspects, the techniques described herein relate to a cleaning device, wherein the flexible cleaner includes one or more circular disks spaced along the rod.

In some aspects, the techniques described herein relate to a cleaning device, wherein the rod includes one or more braided strands.

In some aspects, the techniques described herein relate to a cleaning device, further including a tip attached to the distal end of the rod, wherein the tip is spherical.

In some aspects, the techniques described herein relate to a cleaning device for cleaning a channel of a medical device, the cleaning device including: a rod; a bristles positioned around and oriented radially outward from the rod; a cover proximal of the bristles and including a anchor points; a propeller fixedly coupled to the rod, and rotatably coupled to the cover radially inward of the cover; and a pull cord attached to the anchor points.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 depicts a view of an exemplary brush for use in an endoscope, according to aspects of this disclosure.

FIG. 2 depicts a view of an exemplary brush positioned within an endoscope, according to aspects of this disclosure.

FIG. 3 depicts an exploded view of an assembly of an exemplary brush, according to aspects of this disclosure.

FIG. 4 depicts a side view of a cover of an exemplary brush, according to aspects of this disclosure.

FIG. 5 depicts a view of an exemplary brush with disposable disks for cleaning an endoscope, according to aspects of this disclosure.

FIGS. 6 and 7 depict views of an exemplary connection for a brush with a reusable wire and disposable disks for cleaning an endoscope, according to aspects of this disclosure.

FIG. 8 depicts a view of an exemplary brush with a disposable helical structure for cleaning an endoscope, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of +10% in a stated numeric value or range.

Aspects of this disclosure relate to improved cleaning devices for cleaning medical devices such as inner channels of medical devices, including for example endoscopes. As discussed above, endoscopes are currently widely used devices for both diagnosis and treatment of various disorders. But because endoscopes come into contact with various body fluids (e.g., blood and mucous), cleaning is important.

During cleaning, an endoscope is typically immersed in a solution of enzymatic soap and water. The solution is then pumped into the channels and internal mechanical cleaning of the channels is performed using a brush. For example, a technician inserts a cleaning brush from the distal end to the proximal end of the endoscope's working channel. The brush is moved several times bi-directionally from the distal end to the proximal end (back and forth), to assist with the cleaning and removal of debris from the internal channels.

In a first example of this disclosure, a cleaning brush having a propeller is disclosed. In addition to linear movement as the brush is moved through an inner channel of an endoscope by a technician, the brush also cleans the inner channel by rotating the bristles of a brush head of the cleaning brush. This rotational movement is generated by the propeller from the linear motion. The propeller, when moved through fluid, turns a rod of the brush head, which in turn rotates the bristles of the brush head that are connected to the rod.

In a second example of this disclosure, a single-use cleaning brush head (a removable portion) releasably connects with a reusable actuation wire (a reusable portion). The reusable portion may be retained for subsequent use with another brush head/removable portion, while the used brush head/removable portion may be discarded. The removable portion may include circular disks, washers, or other structure that extend to at least a diameter of the inner channel and perform the cleaning. Even though the removable portion is discarded, having the reusable portion reduces cost and material waste. Due to a tight fit between the endoscope channels and the disks/washers, only one passage may be needed.

Disclosed devices may be used to clean endoscopes. Endoscopes typically have one or more working channels (inner channels) through which instrumentation may be passed through for performing diagnostic or therapeutic procedures. The working channel extends from the proximal end (e.g., near the operator of the endoscope) through to the distal end (first placed into a patient's body). After insertion through the working channel, instruments may come into contact with bodily fluids and tissue. Consequently, when the instruments are retracted, the working channel becomes contaminated and needs cleaning. Cleaning the working channel typically involves applying cleaning fluid and/or cleaning devices such as brushes to clear any debris and to sanitize the inner channel, as described above.

An endoscope may be part of a system. For example, an endoscope system may include an endoscope and various system components such as a controller, a light source, a source of suction and/or irrigation, etc. The endoscope may include a handle assembly and a flexible tubular shaft. The handle assembly may include a biopsy port, a biopsy cap, an image capture button, an elevator actuator (if the endoscope is a duodenoscope), locking levers, control knobs, a suction button, an air/water button, a handle body, and an umbilicus. The umbilicus may extend from handle body to one or more auxiliary devices, water/fluid supply, and/or vacuum source. The umbilicus therefore may transmit signals between the endoscope and a controller, to control lighting and imaging components of the endoscope and/or receive image data from the endoscope. The umbilicus also can provide fluid for irrigation from the water/fluid supply and/or suction to a distal tip of the shaft. All of the actuators, elevators, knobs, buttons, levers, ports, or caps of the endoscope may serve any purpose and are not limited by any particular use that may be implied by the respective naming of each component used herein. The endoscope may include various control valves for suction and fluid supply (e.g., air and water), respectively.

The endoscope shaft may terminate at a distal tip. The shaft may include an articulation section for deflecting the distal tip in up, down, left, and/or right directions. Various knobs may be used for controlling such deflection, and various locking levers may lock the knobs in desired positions. A handle body may be tapered and may narrow as the handle extends distally such that the profile of handle body is smaller at its distal end than at its proximal end.

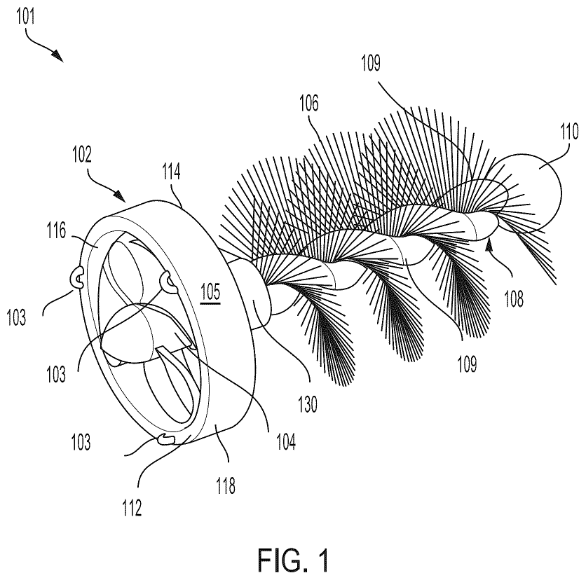

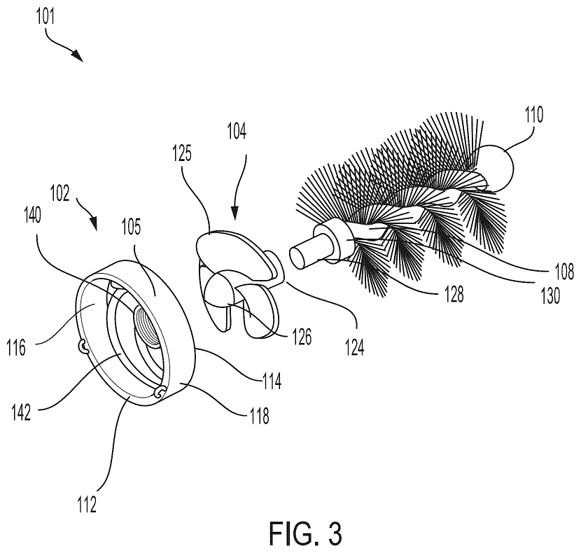

As discussed, certain aspects relate to improved cleaning devices for use in endoscopes. For instance, in a first example of this disclosure, a cleaning brush that can use rotational movement to clean an endoscope working channel is discussed further with respect to FIGS. 1-3.

Turning now to the figures, FIGS. 1-3 depict views of an exemplary brush 100 for use in cleaning an endoscope working channel, according to aspects of this disclosure. As explained further herein, brush 100 may be used to clean an inner channel of an endoscope with both rotational and linear movement.

As depicted, cleaning brush 100 includes a brush head 101 and a pull wire/cord 122. Brush head 101 is shown in FIG. 1 and includes a cover 102, a propeller 104, bristles 106, and a rod 108 having a tip 110. But cleaning brush 100 may have other components. Further, some components may be omitted from some aspects.

Rod 108 may be flexible, extend along an axis of brush head 101, and connect to propeller 104. Rod 108 may be made of rubber or other suitable biocompatible material. Rod 108 may be formed of a braided material such as two braids (braided strands) 109. In other cases, rod 108 may be a single, straight strand. Tip 110 may be positioned at a distal end of rod 108. Tip 110 is distal of the distalmost end of the bristles 106, spherical, and formed of a soft material such as rubber that will not scratch the inner channel of the endoscope. Bristles 106 are attached to braids 109 of rod 108, and positioned around and oriented radially outward from rod 108. Bristles 106 are in a helical arrangement about rod 108 and wound over three times about rod 108, but any other suitable arrangement may be used. Bristles 106 have radially inward ends that are wedged between the two strands 109. Bristles 106 are flexible and may be made of polypropylene to avoid scratching an inner surface of the endoscope channel.

Rod 108 may be attachable to, and removable from, propeller 104. For example, rod 108 may include a flange 130 and a post 128 at its proximal end. Post 128 inserts into a channel of a cylindrical, tube-like axle 124 at the distal end of propeller 104. Post 128 may be fixed within axle 124 via adhesive or a friction fit, so that propeller 104 and rod 108 rotate together about their axes. Propeller 104 includes axle 124 and blades 125. Axle 124 includes a proximal nose 126 that has a proximal most apex. Nose 126, which is conical in shape, may lower resistance when the brush 100 is pulled through the inner channel.

While propeller 104 depicts three blades 125, any number of blades 125 is possible. However, three blades 125 may result in a maximum collision surface area while minimizing the number of blades 125 required. In so doing, such an approach reduces material failures and increases propeller stability while increasing manufacturability of propeller 104.

Propeller 104 may be rotatably affixed within cover 102 via a bearing 140. Bearing 140 is tube-like with an inner diameter greater than the outer diameter of axle 124. Axle 124 is within, and rotatable relative to, bearing 140. Blades 125 prevent proximal movement of cover 102 relative to propeller 104 and rod 108. A proximal face of flange 130 is adjacent a distal end of bearing 140 and prevents distal movement of cover 102 relative to propeller 104 and rod 108.

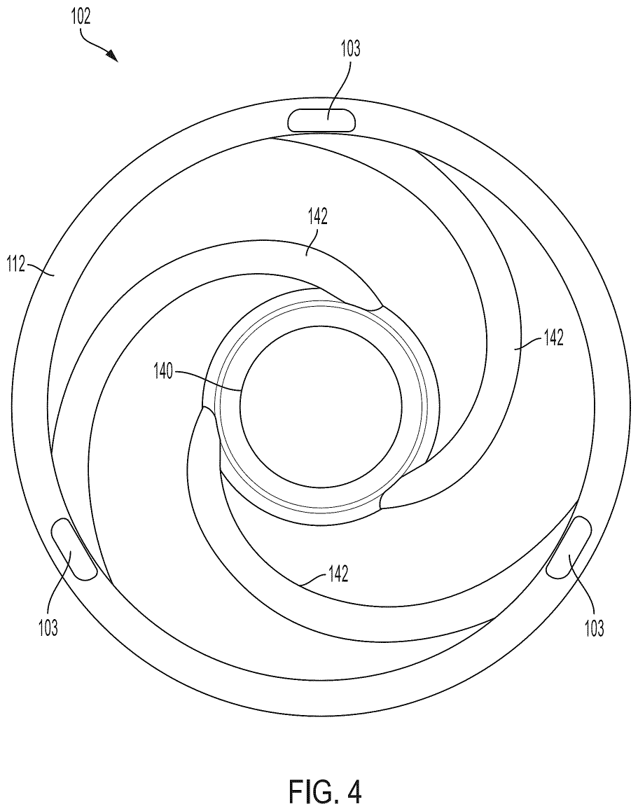

Cover 102 includes an annular disk 105 surrounding bearing 140 and attached to bearing 140 by one or more spokes 142, as shown in FIG. 4. Cover 102 has a proximal edge 112 (proximal of blades 125), a distal edge 114 (distal of blades 125), an inner surface 116, and an outer surface 118. Inner surface 116 is oriented towards propeller 104, whereas outer surface 118 is oriented towards the channel of the endoscope when brush 100 is in use.

Each spoke 142 has a radially outer end attached to the inner surface 116 and a radially inner end attached to bearing 140. The spokes 142 may be curved or straight. Cover 102 serves to isolate propeller 104 from the inner surface of the channel, thereby preventing propeller 104 from contact with the channel walls of the endoscope.

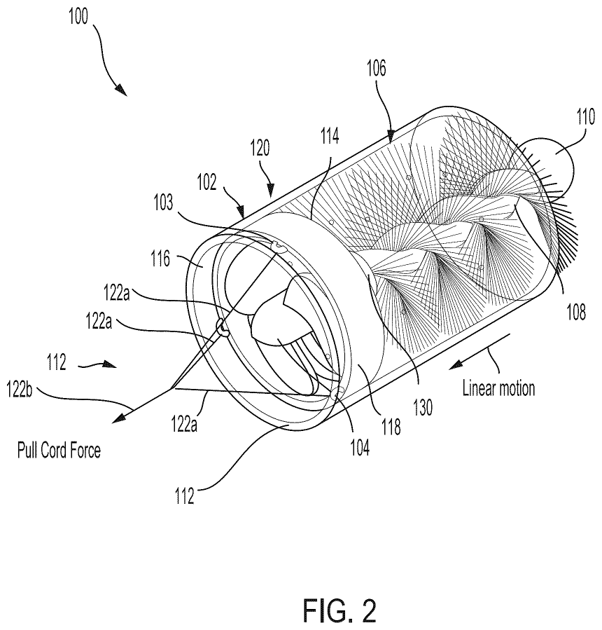

Proximal edge 112 includes one or more anchors 103, in this case three anchors 103 equally spaced around the circumference of proximal edge 112. But any number of anchors 103 is possible. Each anchor 103 includes an opening for attachment of a pull wire/cord 122 (see FIG. 2). Pull cord (member) 122 includes three wires (segments) 122a, each having a distal end attached to an anchor 103. The proximal ends of wires 122a attach to one another. Pull cord wire 122b, proximal of that attachment point, is a single wire or cord, for example, three wires wound together that separate into the wires 122a. While three anchors are depicted, any number of anchors is possible. In a typical configuration, however, a number of anchors 103 equals a number of wires 122a, to achieve a balance in pull force.

In some aspects, a combined diameter of the bristles 106 and rod 108 may be slightly greater than a diameter of the endoscope channel, and an outer diameter of cover 102 is less than the diameter of the endoscope channel. In some aspects, cleaning brush 100 may include brush elements that are of varying size, rigidity, and/or material, thereby further improving cleaning performance.

Brush 100 may be used to clean an inner channel of an endoscope with both rotational and linear movement. For instance, FIG. 2 depicts a view of an exemplary brush 100 positioned within an endoscope channel, according to aspects of this disclosure. In the example depicted, brush 100 is positioned (e.g. by a technician) within channel wall 120 of an endoscope and is attached to pull cord 122, which facilitates cleaning of the inner channel of an endoscope.

Continuing the example, the technician may use pull cord 122 to pull brush 100 through the endoscope channel (e.g., from the proximal end and to the distal end of the endoscope). In so doing, the linear movement pushes internal debris and particles along its path. Additionally, as the brush 100 moves through the endoscope, the propeller 104 rotates, thereby causing the rod 108 to rotate and the bristles 106 to rotate, cleaning an inside surface of the endoscope channel.

This rotational motion is achieved, for example, by using a static fluid (e.g., cleaning fluid) with an active moving propeller 104. The linear kinetic energy from dragging motion makes the fluid within the channel collide with the propeller blade's surface, which in turn generates rotation and an improved cleaning process inside the working channel. When rod 108 is caused to rotate by propeller 104, bristles 106 also rotate, cleaning the inner channel of the endoscope as the brush 100 is moved through the inner channel. This approach increases mechanical action of bristles 106 in removing residues present on the channel, maximize enzymatic soap fluid contact surface area.

To ensure propeller stability, and rotational and linear motion, an equal number of pull cord wires 122a (and therefore anchors 103) and blades 125 (for example, three) is maintained. While increasing the number of pull cord wires 122a may improve force distribution, it may also negatively impact the fluid dynamics of water before it reaches propeller 104. For instance, three wires 122a may be attached at equally spaced 120 degree intervals throughout the propeller cover 102, to be dragged along the endoscope working channel.

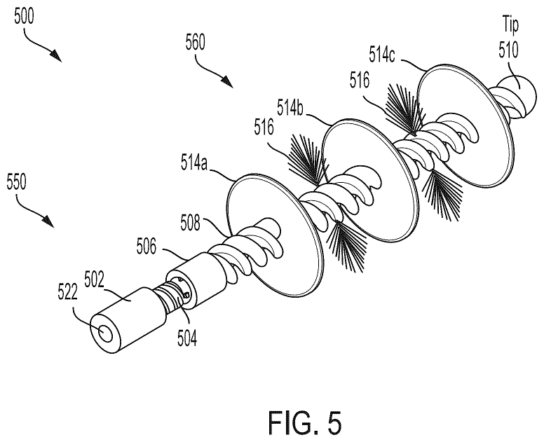

In a second example of this disclosure, a single-use cleaning brush with a reusable portion is discussed with respect FIGS. 5-7. FIGS. 5-7 depict views of an exemplary brush 500 with disposable disks for cleaning an endoscope working channel, according to aspects of this disclosure. Brush 500 includes ferrule 502, connector screw 504, connector 506, rod 508, and a flexible cleaner such as one or more circular disks 514a-c positioned around rod 508, and tip 510.

Rod 508 and tip 510 may have any of the characteristics of those elements in other disclosed embodiments. Rod 508 may be flexible and extend along an axis of the brush 500. Rod 508 may be made of rubber or other suitable biocompatible material. Rod 508 may be formed of a braided material such as two or more braided strands. In other cases, rod 508 may be a single, straight strand. Rod 508, as best shown in the figures, may be a single coiled strand with spacing between adjacent coils. Rod 508 is fixedly connected to connector 506 on the proximal end and tip 510 on the distal end. Tip 510 can have similar structure and/or functionality as tip 110 discussed above.

Bristles 516 may be attached to rod 508, positioned around rod 508, and oriented radially outward from rod 508. Bristles 516 may be positioned between the disks 514a-c. For example, bristles 516 may be positioned between disks 514a and 514b and between disks 514b and 514c, and so forth. Bristles 516 may have any of the characteristics described for bristles throughout this disclosure.

Disks 514a-c are positioned around rod 508 and spaced apart from each other. While three disks 514a-c are shown, any number is possible. In an example, disks 514a-c are formed of rubber or other flexible material. In one example, disks 514a-c are spaced at equal intervals, with approximately 7/16 inches of space between adjacent disks 514a-c. In another example, the disks are approximately 1.05% of a diameter of the inner channel of the endoscope, ensuring a tight fit within the endoscope channel.

Connector 506 is cylindrical/tube-like, with an inner channel 520. One or more teeth 505 are within channel 520 at a proximal end of channel. The embodiment shown in the Figures includes two teeth 505 equally spaced within channel 520 (i.e about 180 degrees apart.

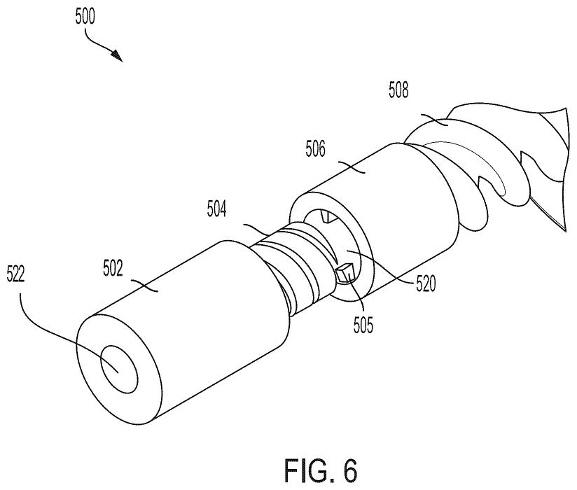

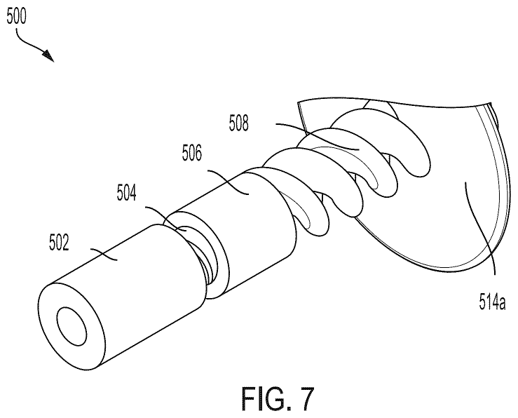

Ferrule 502 may be cylindrical in shape with a channel 522. Ferrule 502 may fixedly attach to, or otherwise be integral with, connector screw 504 on the distal side and may attach to another device such as a pull wire (not shown) on the proximal side. The pull wire may fixedly insert within channel 522 via adhesive, friction fit, or other suitable structure. Connector screw 504 is a threaded connection, for example, a coil having spaces between adjacent turns of the coil. Connector screw 504 may couple with teeth 505 of connector 506 by inserting teeth within the spaces and rotating ferrule 502 relative to connector 506, until connector 506 abuts ferrule 502. FIGS. 5 and 6 show ferrule 502 and connector 506 unconnected, while FIG. 7 shows them in a partially connected state. Each of ferrule 502, connector screw 504, teeth 505, and connector 506 may be formed of plastic, metal, or other suitable material.

To clean an inner channel of an endoscope, brush 500 is inserted into one end of the inner working channel of the endoscope and pulled out from the other end. The disks 514a-c clean the channel and remove debris and other bodily substances out of the working channel of the endoscope. In some cases, only a single passage through the channel may be needed. When brush 500 is placed within the inner channel, disks 514a-c create a suction space, generating a flow of liquid inside the endoscope to remove the debris. As sometimes bristles themselves leave residue, by not having bristles, brush 500 may leave less residue deposited on the inside of the channel.

Reusable portion 550 and removable portion 560 may be connected and/or disconnected, as described above. Reusable portion 550 includes ferrule 502. connector screw 504, and the pull wire, whereas removable portion 560 includes connector 506, rod 508, disks 514a-c, and tip 510. Connector screw 504 facilitates connection to and disconnection from the removable portion 560.

For example, as depicted in FIG. 6 and described above, connector 506 is disconnected from ferrule 502. Connector 506 includes teeth 505 (or pins) which connect with the connector screw 504, thereby connecting the connector 506 with ferrule 502.

FIG. 7 depicts an example in which reusable portion 550 and removable portion 560 are at least partially connected. As depicted, connector screw 504 is inserted into connector 506, thereby attaching ferrule 502 to connector 506. This connection is made by rotating teeth 506 between the turns of connector screw 504, for example until teeth 505 engage a stop (not shown) that may limit further rotation, or until a proximal face of connector 506 abuts a distal face of ferrule 502.

In some cases, teeth 505 are configured for single use. For example, sufficient rotation and applied force may break teeth 505 from connector 506, allowing connector 506 to release from ferrule 502, helping ensure the removable portion 560 may not be reused. In an example, after a technician completes a cleaning procedure, the technician may further rotate connector screw 504 relative to connector 506, in the same direction as when the two parts were connected, thereby breaking teeth 505 against the stop.

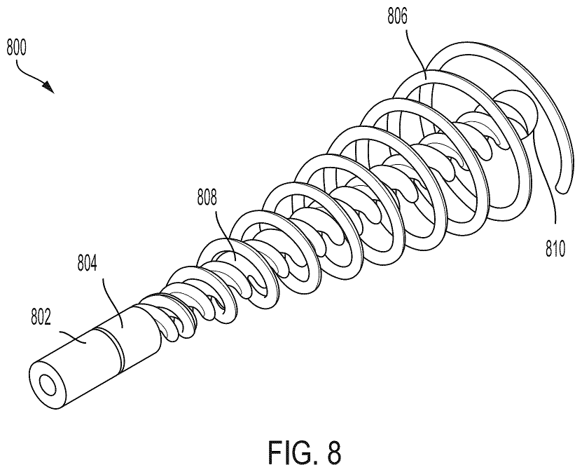

FIG. 8 depicts a view of an exemplary brush 800 for cleaning an endoscope, according to aspects of this disclosure. Brush 800 includes wire (not shown) that connects to ferrule 802, connector 804, rod 808, tip 810, and a flexible cleaner such as helical coil 806. The wire, ferrule 802, connector 804, rod 808, and tip 810 may have any of the aspects discussed with respect to brush 500 shown in FIGS. 5-7.

As depicted, coil 806 has a conical and helically wound about rod 808. Coil increases in diameter from the proximal end to the distal end. Helical coil 806 may be formed of polypropylene, ABS, polyethylene, or other flexible suitable material, to ensure that it can support being exposed to a disinfectant fluid and not damage the endoscope working channel. To clean an inner channel of an endoscope, the proximal end of brush 800 may be inserted first, and brush 800 pulled through the inner channel.

Helical coil 806 may be expandable and compressible. Therefore, when being dragged through the inner channel, helical coil 806 may compress, thereby achieving a tight fit within the channel and improved cleaning. In some cases, the diameter of the helical coil 806, expanded, may be approximately 1.5% of the diameter of the endoscope working channel. This helps to ensure that a change of the geometry based on the material may generate pressure over the inner surface of the endoscope working channel, removing the heaviest debris that may have stuck to the endoscope.

Although the term endoscope may be used herein, it will be appreciated that other devices, including, but not limited to, other types of scopes, endoscopes, and medical devices, such as cholangioscopes, duodenoscopes, colonoscopes, ureteroscopes, bronchoscopes, laparoscopes, sheaths, catheters, or any other suitable delivery device or medical device, may be used in connection with the devices of this disclosure, and the devices, systems, and methods discussed below may be incorporated into any of these or other medical devices.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A cleaning device for cleaning a channel of a medical device, the cleaning device comprising:
a rod;
a plurality of bristles positioned around and oriented radially outward from the rod;
a cover proximal of the plurality of bristles;
a bearing radially inward of an inner surface of the cover; and
a propeller coupled to the rod, positioned radially inward of the cover, and rotatable within the bearing.

2. The cleaning device of claim 1, further comprising a flange and a post, wherein the post is affixed to the rod via the flange, and wherein the propeller includes an axle that is configured to receive the rod.

3. The cleaning device of claim 1, wherein the bearing is affixed to an inner surface of the cover via one or more spokes.

4. The cleaning device of claim 1, wherein the plurality of bristles is formed of polypropylene.

5. The cleaning device of claim 1, wherein each of the plurality of bristles extends radially with a first diameter that is greater than a second diameter of the cover.

6. The cleaning device of claim 1, wherein the rod includes one or more braided strands.

7. The cleaning device of claim 1, further comprising a pull cord that extends proximally from the cover and a plurality of anchor points positioned on the cover, wherein the plurality of anchor points is connected to the pull cord.

8. The cleaning device of claim 7, wherein the plurality of anchor points comprises three anchor points, and wherein the pull cord comprises three segments, wherein each segment is connected to a respective anchor point.

9. The cleaning device of claim 1, wherein the propeller includes a nose at a proximal edge of the propeller.

10. The cleaning device of claim 1, wherein the propeller is configured such that, when a force is applied in a proximal direction in a presence of a fluid, the propeller rotates, thereby causing the rod and the plurality of bristles to rotate.

11. The cleaning device of claim 1, further comprising a tip at a distal end of the rod.

12. The cleaning device of claim 11, wherein the tip is spherical and formed of rubber.

13. A cleaning device for cleaning a channel of a medical device, the cleaning device comprising:
a rod;
a connector at a proximal end of the rod;
a flexible cleaner positioned around the rod;
a ferrule configured to removably connect with the connector; and
a pull member connected to the ferrule.

14. The cleaning device of claim 13, further comprising a tip at a distal end of the rod, wherein the ferrule is fixedly attached to a connector screw, wherein the rod, the connector, and the flexible cleaner, are removably connected to the ferrule and the connector screw.

15. The cleaning device of claim 13, wherein the flexible cleaner comprises a helical coil attached to the rod, wherein the helical coil comprises a first diameter at a proximal end of the rod and a second diameter at a distal end of the rod, wherein the second diameter is greater than the first diameter.

16. The cleaning device of claim 13, wherein the ferrule is attached to a connector screw, and wherein the connector comprises one or more teeth that interface with the connector screw.

17. The cleaning device of claim 13, wherein the flexible cleaner comprises one or more circular disks spaced along the rod.

18. The cleaning device of claim 15, wherein the rod includes one or more braided strands.

19. The cleaning device of claim 15, further comprising a tip attached to the distal end of the rod, wherein the tip is spherical.

20. A cleaning device for cleaning a channel of a medical device, the cleaning device comprising:
a rod;
a plurality of bristles positioned around and oriented radially outward from the rod;
a cover proximal of the plurality of bristles and including a plurality of anchor points;
a propeller fixedly coupled to the rod, and rotatably coupled to the cover radially inward of the cover; and
a pull cord attached to the plurality of anchor points.
